(19) United States
(12) Patent Application Publication
(10) Pub. No.: US 2024/0068960 A1
(43) Pub. Date: Feb. 29, 2024

(54) MEASUREMENT SYSTEM AND FLOAT

(71) Applicant: NEC Corporation, Tokyo (JP)

(72) Inventor: Taichi Tanaka, Tokyo (JP)

(73) Assignee: NEC Corporation, Tokyo (JP)

(21) Appl. No.: 18/230,523

(22) Filed: Aug. 4, 2023

(30) Foreign Application Priority Data
Aug. 25, 2022 (JP) 2022-134142

(51) Int. Cl. G01N 22/00; G01F 23/64; G01F 23/76; G01N 33/18

(52) U.S. Cl. CPC G01N 22/00; G01F 23/64; G01F 23/76; G01N 33/1886

(57) ABSTRACT

A measurement apparatus of the present disclosure includes a measuring unit configured to acquire the reflected wave of a radio wave applied to a float and measure the state of a liquid based on the intensity of the acquired reflected wave. Then, the float is configured to float in a liquid in a manner such that a floating height relative to the liquid changes in accordance with the state of the liquid, and is further configured in a manner such that the intensity of the reflected wave of an applied radio wave changes in accordance with a change in the floating height relative to the liquid.

### INCORPORATION BY REFERENCE

This application is based upon and claims the benefit of priority from Japanese patent application No. 2022-134142, filed on Aug. 25, 2022, the disclosure of which is incorporated herein in its entirety by reference.

### TECHNICAL FIELD

The present invention relates to a system that measures the state of a liquid and to a float used therein.

### BACKGROUND ART

Patent Literature 1 describes a method for measuring the salinity of salt water in a salt field. First, as a general method for measuring salt water concentration, a method using a hydrometer, or the Baume scale, is described. However, this method requires collection of salt water samples from a salt field, and it is difficult to collect the samples because the area of the salt field is large. Thus, Patent Literature 1 describes a problem that frequent observation of the salt water concentration becomes difficult and the amount of salt produced varies greatly.

Patent Literature 1 also describes, as another method for measuring salinity, using a new device based on the principle of buoyancy. Specifically, the device has a hemispherical main body and a graduated bar provided on top of the main body and, when a desired salinity is reached in salt water, floats up to a corresponding scale. Therefore, by observing the scale of the device floating in a salt field from a distance, the salt water concentration is measured.
Patent Literature 1: Japanese Translation of PCT International Application Publication No. JP-T 2006-511795

However, the abovementioned technique described in Patent Literature 1 has problems that the salinity cannot be measured with high accuracy and that it takes time and effort for the measurement. Many of the salt fields have an area of several square kilometers, and some have a more area. Therefore, in a case where the abovementioned devices are floated in such a wide-area salt field, there is a need to observe the scales of the devices from a distance, or to move closer to the individual devices to observe. Then, in the case of observing the devices from a distance, it is difficult to observe the scales with high accuracy. On the other hand, in the case of moving closer to the individual devices to observe the scales, it takes time and effort for the movement. As a result, there arises a problem that the salinity cannot be measured easily and accurately. Moreover, such a problem may occur not only when measuring the salinity of salt water in a salt field, but also when measuring the state of a liquid such as the concentration of any liquid existing in any place.

### SUMMARY OF THE INVENTION

Accordingly, an object of the present disclosure is to provide a system that measures the state of a liquid easily and accurately.

A measurement system as an aspect of the present disclosure includes: a float configured to float in a liquid in a manner such that a floating height relative to the liquid changes in accordance with a state of the liquid; and a measuring unit configured to measure the state of the liquid based on intensity of a reflected wave of a radio wave applied to the float. The float is configured in a manner such that the intensity of the reflected wave changes in accordance with a change in the floating height relative to the liquid.

A float as an aspect of the present disclosure is configured to float in a liquid in a manner such that a floating height relative to the liquid changes in accordance with a state of the liquid, and is further configured to reflect an applied radio wave in a manner such that intensity of a reflected wave changes in accordance with a change in the floating height relative to the liquid.

A measurement method as an aspect of the present disclosure includes acquiring a reflected wave of a radio wave applied to a float and measuring a state of a liquid based on intensity of the acquired reflected wave. The float is configured to float in a liquid in a manner such that a floating height relative to the liquid changes in accordance with the state of the liquid, and is further configured in a manner such that the intensity of the reflected wave of an applied radio wave changes in accordance with a change in the floating height relative to the liquid.

A measurement apparatus as an aspect of the present disclosure includes a measuring unit configured to acquire a reflected wave of a radio wave applied to a float and measure a state of a liquid based on intensity of the acquired reflected wave. The float is configured to float in the liquid in a manner such that a floating height relative to the liquid changes in accordance with the state of the liquid, and is further configured in a manner such that the intensity of the reflected wave of the applied radio wave changes in accordance with a change in the floating height relative to the liquid.

A computer program as an aspect of the present disclosure includes instructions for causing a computer to execute processes to acquire a reflected wave of a radio wave applied to a float and measure a state of a liquid based on intensity of the acquired reflected wave. The float is configured to float in the liquid in a manner such that a floating height relative to the liquid changes in accordance with the state of the liquid, and is further configured in a manner such that the intensity of the reflected wave of the applied radio wave changes in accordance with a change in the floating height relative to the liquid.

With the configurations as described above, the present disclosure enables easy and highly accurate measurement of the state of a liquid.

### BRIEF DESCRIPTION OF DRAWINGS

FIG. 1 is a block diagram showing the overall configuration of a measurement system in a first example embodiment of the present disclosure;

FIG. 2 is a view showing an example of the configuration of a float disclosed in FIG. 1;

FIG. 3 is a view showing an example of the configuration of the float disclosed in FIG. 1;

FIG. 4 is a view showing an example of the configuration of the float disclosed in FIG. 1;

FIG. 5 is a view showing an example of the configuration of the float disclosed in FIG. 1;

FIG. 6 is a view showing an example of the configuration of the float disclosed in FIG. 1;

FIG. 7 is a view showing an example of the configuration of the float disclosed in FIG. 1;

FIG. 8 is a block diagram showing the configuration of a measurement apparatus disclosed in FIG. 1;

FIG. 9 is a flowchart showing the operation of the measurement apparatus disclosed in FIG. 1;

FIG. 10 is a view showing an example of the configuration of a float used in a measurement system in a second example embodiment of the present disclosure;

FIG. 11 is a view showing the example of the configuration of the float disclosed in FIG. 10;

FIG. 12 is a view showing an example of the configuration of a float used in a measurement system in a third example embodiment of the present disclosure;

FIG. 13 is a view showing an example of the configuration of a float used in a measurement system in a fourth example embodiment of the present disclosure;

FIG. 14 is a view showing the example of the configuration of the float disclosed in FIG. 13;

FIG. 15 is a block diagram showing the hardware configuration of a measurement apparatus in a fifth example embodiment of the present disclosure; and

FIG. 16 is a block diagram showing the configuration of the measurement apparatus in the fifth example embodiment of the present disclosure.

### EXAMPLE EMBODIMENT

### First Example Embodiment

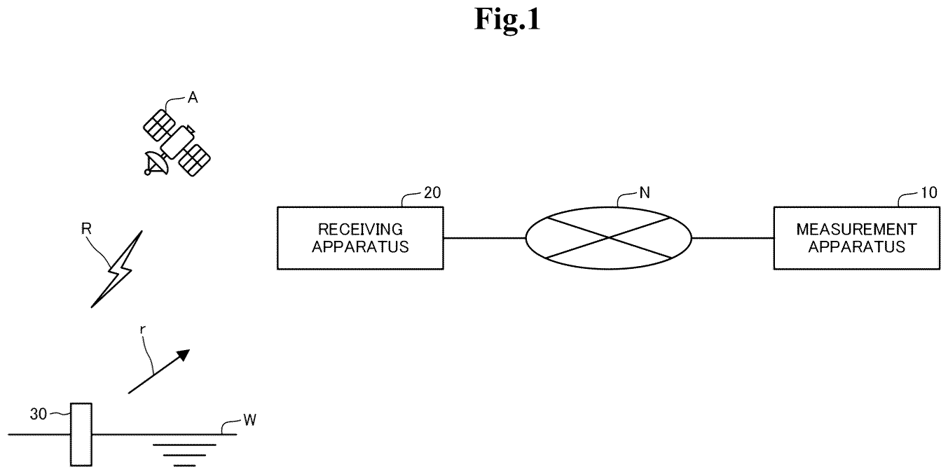

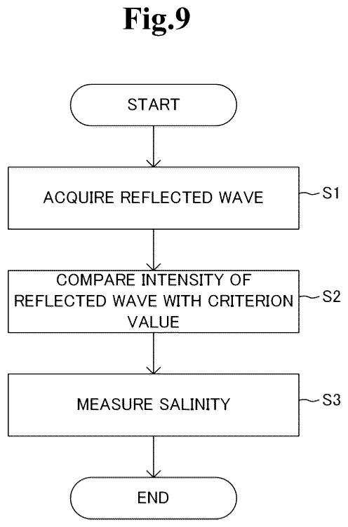

A first example embodiment of the present disclosure will be described with reference to FIGS. 1 to 9. FIGS. 1 to 8 are views for describing the configuration of a measurement system, and FIG. 9 is a view for describing the processing operation of the measurement system.

### Configuration

The measurement system in this example embodiment is for measuring the salinity of salt water in a salt field. In particular, the measurement system in this example embodiment is suitable for measuring the salinity of salt water in a solar saltern where crystallized salt is produced by evaporating and concentrating water containing salt such as seawater and water of a salt lake. For example, a salt field where the measurement system is used is a place having an area of several square kilometers or more, but may be used in a salt field of any size.

The measurement system of the present disclosure is not limited to being used for measuring the salinity of salt water in a salt field, and can be applied to measuring the concentration of any liquid existing in any place. For example, the measurement system may be applied to measuring the salinity of a liquid existing in a lake, sea and the like, or may be applied to measuring the concentration of a colloidal solution such as mud. Although the measurement target liquid is, for example, a solution in which a solute is dissolved in a solvent and the concentration is the ratio of the solute in the solution, the concentration of any substance molted in any liquid may be measured. In addition, the measurement place may be any place.

Further, the measurement system of the present disclosure is not limited to measuring the concentration of a liquid, and can also be applied to measuring the temperature and components of a liquid. That is to say, the measurement system of the present disclosure can also be applied to measuring the state of a liquid, such as the concentration, temperature and components of a liquid.

As shown in FIG. 1, the measurement system in this example embodiment includes a float that floats in salt water W (liquid) in a salt field, a receiving apparatus 20 that receives a reflected wave f of a radio wave from the float 30, and a measurement apparatus 10 that measures the salinity of the salt water W. In this example embodiment, a synthetic aperture radar (SAR) mounted on an artificial satellite A that applies a radio wave R to the float 30 as will be described later is used for measurement of the salinity of the salt water W. However, the radio wave R applied to the float 30 is not limited to a radio wave applied by the synthetic aperture radar, and may be the radio wave R applied by any apparatus. For example, the radio wave R emitted by a flying object such as an artificial satellite composing a GNSS (Global Navigation Satellite System) and an aircraft may be used, or the radio wave R emitted by an apparatus that is installed on the ground and emits a radio wave may be used. Below, the respective components will be described in detail.

The float 30 is a floating object placed in the salt field, and is composed of a member having buoyancy that floats in the salt water W in the salt field. For example, the floats 30 are placed at a plurality of locations to measure concentrations in the vast salt field.

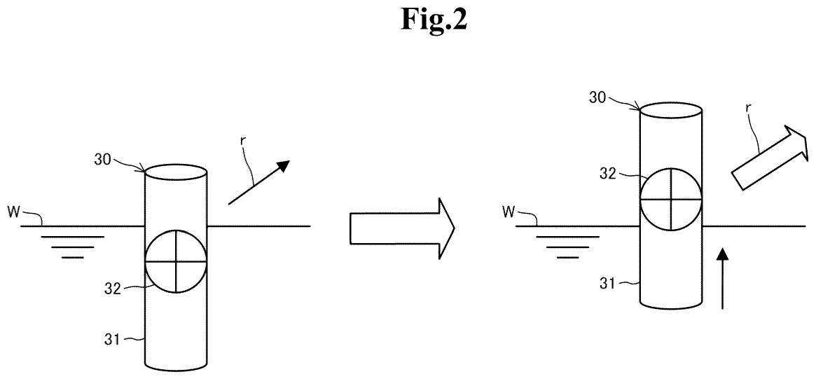

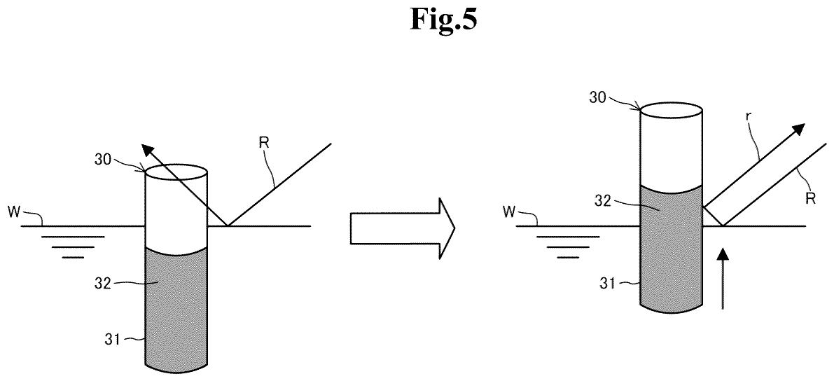

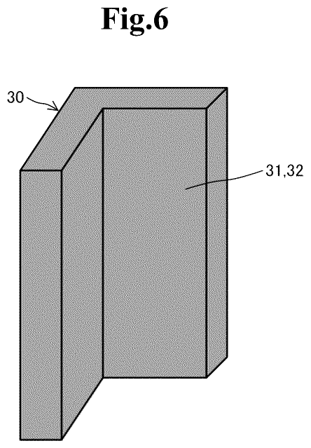

The float 30 includes, as shown in FIG. 2, a float main body 31 that is a floating object floating under the buoyancy of the salt water W, and a reflecting part 32 that reflects the radio wave R applied thereto. As an example, the float main body 31 of the float 30 in the example of FIG. 2 is formed in a cylindrical shape having a predetermined height, and is equipped with the reflecting part 32 at a predetermined height position in the height direction, in this example, at a substantially central position in the height direction. Although the example of FIG. 2 shows a case where the reflecting part 32 is mounted as a separate member on a partial spot of the float 30, a part composed of a conductor surface of the surface of the float main body 31 may be configured as the reflecting part 32 as shown in other configuration examples of the float 30 shown in FIGS. 5, 6 and so forth to be described later. Thus, as shown in FIGS. 5, 6 and so forth, the float main body 31 may be partially or entirely configured as the reflecting part 32.

The float main body 31 is placed so as to float in the salt water W with the height direction of the cylindrical shape located in the vertical direction, and is configured in a manner such that the floating height varies in accordance with the salinity of the salt water W. Specifically, the shape, volume, density, mass and so forth of the float main body 31 are set in a manner such that the floating height relative to the salt water W increases as the salinity of the salt water W increases. For example, the float 30 is configured in a manner such that when the salinity of the salt water W is lower than a specific salinity set in advance, the reflecting part 32 provided near the center in the height direction sinks and is located below the water surface as shown in the left view of FIG. 2. On the other hand, the float 30 is configured in a manner such that when the salinity of the salt water W reaches or exceeds the specific salinity set in advance, the reflecting part 32 provided near the center in the height direction floats up to be located above the water surface as shown in the right view of FIG. 2.

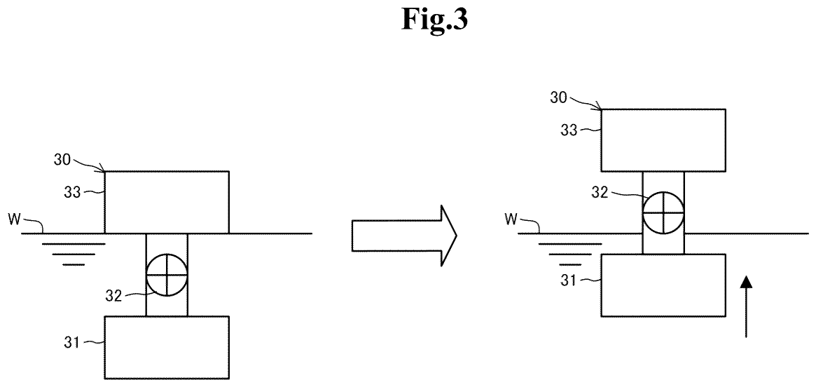

FIG. 3 shows another configuration example of the float 30. As shown in this view, the float 30 may include float main bodies 31 and 33 having large volumes at the lower end and the upper end in the height direction, and include the reflecting part 32 therebetween. The float 30 having such a configuration is also configured in a manner such that when the salinity of the salt water W reaches or exceeds a specific salinity set in advance, the float main body 31 causes the reflecting part 32 to float up to be located above the water surface of the salt water W as shown in the right view of FIG. 3. Thus, by providing the float with the float main body 31 having a large volume at the lower end, the height position of the reflecting part 32 can be moved up and down to a height position corresponding to the salinity in appropriate response to a change in the salinity of the salt water W. Besides, by providing the float with the float body 33 having a large volume at the upper end, the float 30 itself can be prevented from sinking too much when the salinity of the salt water W is low, and the retrieval and maintenance of the float 30 are facilitated.

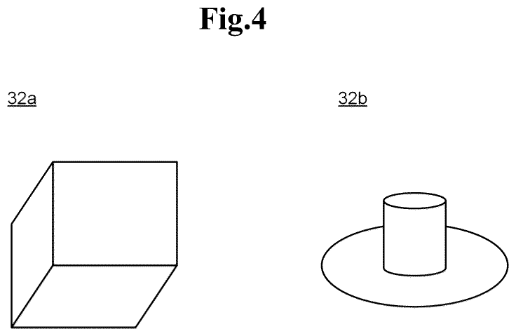

The reflecting part 32 of the float 30 is configured as a device that efficiently reflects a radio wave applied by the artificial satellite A to a direction from which the radio wave has been applied. For example, as indicated by reference numeral 32a in FIG. 4, the reflecting part 32 may be composed of a corner reflector whose three sides are surrounded by reflectors made of conductive surfaces efficiently reflecting electromagnetic waves. Also, as indicated by reference numeral 32b in FIG. 4, the reflecting part 32 may be composed of a structure having a conductive surface and a cylindrical body placed at the center of a disc. Thus, by composing the reflecting part 32 with mutually intersecting conductor surfaces, especially, mutually orthogonal conductor surfaces, strong backscattering to the applied radio wave occurs, and a reflected wave r obtained by reflecting the applied radio wave R can be generated.

By using the float 30 with the configuration as described above, when the salinity of the salt water W is low, the reflecting part 32 is located below the water surface as shown in the left views of FIGS. 2 and 3, and the reflected wave r of the applied radio wave R is not generated, or the reflected wave r with low intensity is generated. On the other hand, when the salinity of the salt water W is high, the reflecting part 32 is located above the water surface as shown in the right views of FIGS. 2 and 3, and the reflected wave r with high intensity of the applied radio wave R is generated.

FIG. 5 shows still another configuration example of the float 30. As shown in this view, the float 30 is formed entirely in a cylindrical shape to compose the float main body 31, and the surface of the float main body 31 downward from a predetermined height position is made of a conductive surface to form the reflecting part 32. The shape, volume, density, mass and so forth of the float main body 31 having such a configuration are also set in a manner such that the floating height relative to the salt water W increases as the salinity of the salt water W increases. Therefore, when the salinity of the salt water W is lower than a specific salinity set in advance, the reflecting part 32 is located below the water surface as shown in the left view of FIG. 5, and the reflected wave r of the applied radio wave R is not generated, or the reflected wave r with low intensity is generated. On the other hand, as the salinity of the salt water W increases, the area of the reflecting part 32 located above the water surface and exposed from the water surface as shown in the right view of FIG. 5 increases, and the intensity of the reflected wave r of the applied radio wave R increases.

FIG. 6 shows a modified example of the shape of the float 30 shown in FIG. 5. As shown in this view, the float 30 may be formed in a substantially L-shape bent along the height direction, and the surface thereof may be composed of a conductor surface. Then, the float 30 is placed so as to float in the salt water W with the inner surface of the bent substantially L-shape facing a direction in which the radio wave R enters. Consequently, as the salinity of the salt water W increases, the area of the reflecting part 32 located above the water surface and exposed from the water surface increases, and the intensity of the reflected wave r of the applied radio wave R increases.

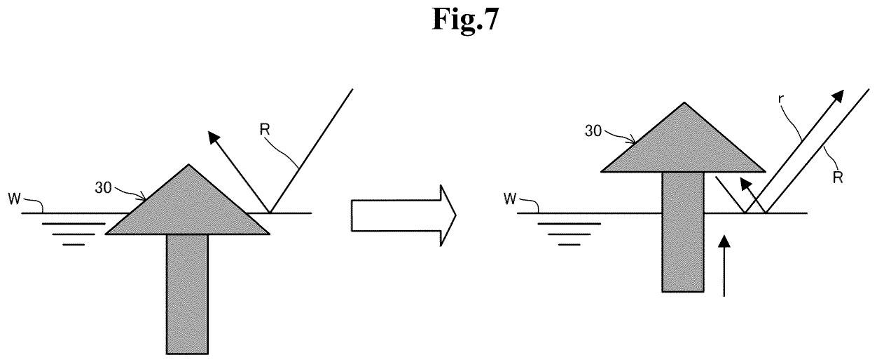

FIG. 7 shows still another configuration example of the float 30. As shown in this view, the float 30 includes the float main body 31 formed in a columnar shape on the lower side in the height direction, and the reflecting part 32 with an umbrella-shaped surface being composed of a conductor surface at the upper end in the height direction. The shape, volume, density, mass and so forth of the float main body 31 having such a configuration are also set in a manner such that the floating height relative to the salt water W increases as the salinity of the salt water W increases. Therefore, when the salinity of the salt water W is lower than a specific salinity set in advance, only an upper portion of the reflecting part 32 is exposed from the water surface as shown in the left view of FIG. 7, so that the reflected wave r of the applied radio wave R is not generated, or the reflected wave r with a low intensity is generated. On the other hand, as the salinity of the salt water W increases, the reflecting part 32 is located above the water surface and exposed from the water surface and, in particular, the lower surface of the umbrella-shaped portion floats above the water surface as shown in the right view of FIG. 7. Then, the applied radio wave R is reflected by the water surface and the lower surface of the umbrella-shape of the reflecting part 32, and the intensity of the reflected wave r increases.

The receiving apparatus 20 is configured by one or a plurality of information processing apparatuses each including an arithmetic logic unit and a memory unit. The receiving apparatus receives the radio wave r obtained by reflection of the radio wave R applied to the float 30 as described above, and transmits information of the radio wave to the measurement apparatus 10. In particular, the receiving apparatus 20 measures the intensity of the received reflected wave r, and transmits the measurement value to the measurement apparatus 10.

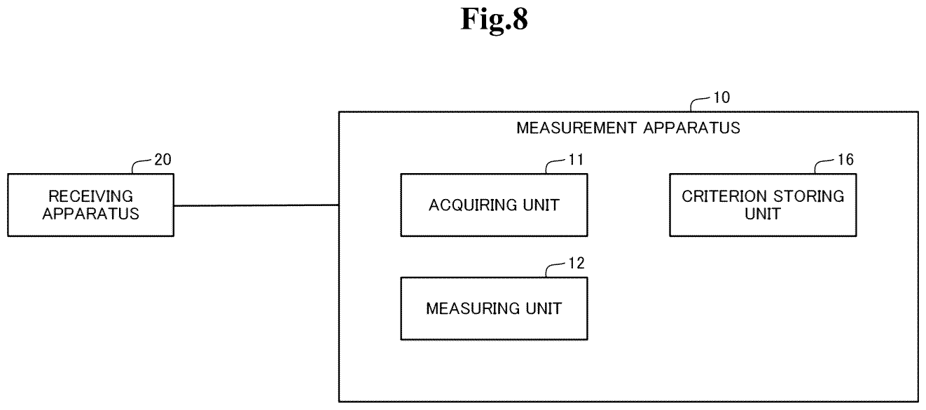

The measurement apparatus 10 is configured by one or a plurality of information processing apparatuses each including an arithmetic logic unit and a memory unit. The measurement apparatus 10 is an information processing apparatus managed by an operator that manages the salinity of the salt water W in the salt field. The measurement apparatus 10 includes an acquiring unit 11 and a measuring unit 12 as shown in FIG. 8. The respective functions of the acquiring unit 11 and the measuring unit 12 can be realized by execution of a program for realizing the respective functions stored in the memory unit by the arithmetic logic unit. The measurement apparatus 10 also includes a criterion storing unit 16. The criterion storing unit 16 is composed of a storage device. Below, each of the components and the operation thereof will be described in detail.

The acquiring unit 11 acquires the intensity of the reflected wave r obtained by reflection of the radio wave R applied to the float 30, transmitted by the receiving apparatus 20. Then, the measuring unit 12 measures the salinity of the salt water W based on the acquired intensity of the reflected wave r. For example, the measuring unit 12 measures the salinity of the salt water W by comparing the value of the acquired intensity of the reflected wave r with a measurement criterion value stored in a criterion storing unit 16.

A specific example of a process of measuring the salinity of the salt water W by the measuring unit 12 will be described. For example, it is assumed that the float 30 has a configuration in which the reflecting part 32 floats up above the water surface when the salinity of the salt water W reaches a specific salinity set in advance as shown in FIG. 2 or 3. In this case, for example, a threshold value of the intensity of the reflected wave r that is a low value such as “0” or “detection limit value” is set as the measurement criterion value in the criterion storing unit 16. Then, the measuring unit 12 checks whether or not the acquired intensity of the reflected waver exceeds the threshold value and, when the threshold value is exceeded, measures that the salinity is equal to or more than the specific salinity set in advance.

Next, for example, a case where the float 30 has a configuration in which as the salinity of the salt water W increases, a floating height at which the reflecting part 32 floats up above the water surface as shown in FIGS. 5 to 7 will be described. In this case, for example, a threshold value, which is the value of the intensity of the reflected wave r that can be generated from the reflecting part 32 when a specific salinity set in advance is reached, is set as a measurement criterion value in the criterion storing unit 16. Then, the measuring unit 12 checks whether or not the acquired intensity of the reflected wave r exceeds the threshold value and, when the threshold value is exceeded, measures that the salinity is equal to or more than the specific salinity set in advance. As another example, in the criterion storing unit 16, a correspondence table between the value of the intensity of the reflected wave r and the salinity may be set as a measurement criterion value. In this case, the measuring unit 12 refers to the correspondence table to identify the value of the salinity corresponding to the value of the acquired intensity of the reflected wave r and determine as the measurement value.

### [Operation]

Next, the operation of the above measurement system will be described mainly with reference to a flowchart of FIG. 9.

The measurement apparatus 10 acquires the intensity of the reflected wave r from the float 30 received by the receiving apparatus 20, for example, at regular time intervals, as an example, at several-hour intervals or one-day intervals (step S1). However, the measurement apparatus 10 is not limited to acquiring the intensity of the reflected wave r at regular time intervals, and may acquire the intensity at any timing on a plurality of dates and times. Moreover, the receiving apparatus 20 from which the measurement apparatus 10 acquires the intensity of the reflected wave r may vary for each acquisition. Therefore, the measurement apparatus 10 may acquire the reflected waves r of the radio waves R applied by different flying objects such as artificial satellites A, from different receiving apparatuses 20, and the locations (orbits) of the receiving apparatuses 20 may be different.

Subsequently, the measurement apparatus 10 compares the acquired intensity of the reflected wave r with a measurement criterion value such as a threshold value stored in the criterion storing unit 16 (step S2). Then, the measurement apparatus 10 measures the salinity of the salt water W based on the result of comparison between the acquired intensity of the reflected wave r and the criterion value (step S3). For example, when the acquired intensity of the reflected wave r exceeds the threshold value, the measurement apparatus 10 measures that the salinity is equal to or more than a specific salinity set in advance. Moreover, for example, with reference to a preset correspondence table of the value of the intensity of the reflected wave r and the salinity, the measurement apparatus 10 identifies the value of the salinity corresponding to the value of the acquired intensity of the reflected wave r, and determines the salinity as the measurement value.

As described above, in the salinity measurement system in this example embodiment, by using the radio wave R applied by the artificial satellite A to measure the intensity of the reflected wave r from the float 30, the salinity of salt water in a salt field can be measured. Therefore, it is possible to measure the salinity with ease and high accuracy even in a salt field having a large area.

Although a case of measuring the salinity of salt water in a salt field has been illustrated above, it can also be applied to measuring the temperature and components of a liquid. In this case, the float 30 described above is composed of a material and with density and so forth that change a floating height with respect to a liquid in accordance with a change in the state of the liquid, such as the temperature and components.

### Second Example Embodiment

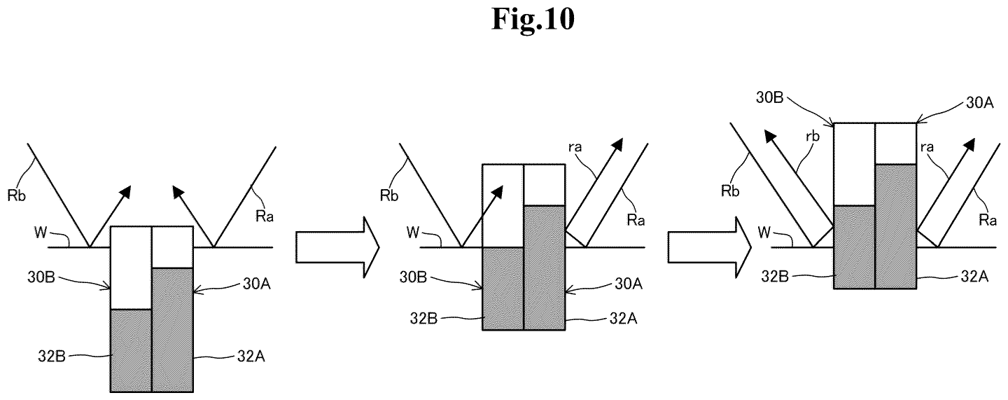

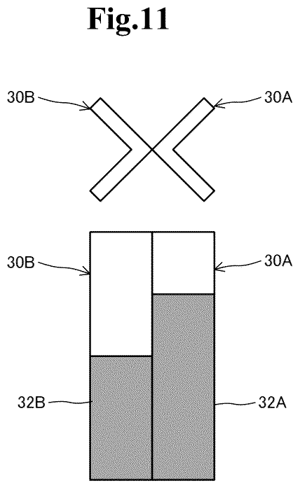

Next, a second example embodiment of the present disclosure will be described with reference to FIGS. 10 and 11. FIGS. 10 and 11 are views for describing the configuration of a float in this example embodiment. In this example embodiment, a different configuration from the above example embodiment will be mainly described.

As shown in FIG. 10, the float in this example embodiment includes two floats, a first float 30A and a second float 30B. For example, the first float 30A and the second float 30B are each formed in a substantially L-shape bent along the height direction as shown in FIG. 6 described above, and have the same shape and density. As shown in FIG. 10, the surfaces of the first float 30A and the second float 30B downward from a predetermined height of the float main bodies are made of conductive surfaces to form reflecting parts 32A and 32B, and height positions at which the reflecting parts 32A and 32B are formed are different from each other. Specifically, the reflecting part 32A of the first float 30A is formed toward the lower end from a higher position than the reflecting part 32B of the second float 30B. Therefore, a float main body (first floating object) of the first float 30A is configured to, when the salinity of the salt water W reaches a preset first salinity (first specific state), float up so that the reflecting part 32A (first reflecting part) is located above the water surface as shown in the center view of FIG. 10. Meanwhile, at this stage, the reflecting part 32B of the second float 30B remains located below the water surface. On the other hand, a float main body (second floating body) of the second float 30B is configured to, when the salinity of the salt water W reaches a second salinity that is a higher value than the first salinity (second specific state), float up so that the reflecting part 32B (second reflecting part) is located above the water surface as shown in the right view of FIG. 10.

Further, the first float 30A and the second float 30B, which compose the float in this example embodiment, are arranged so as to reflect radio waves applied from mutually different directions. Specifically, the first float 30A and the second float 30B are each formed in a substantially L-shape bent along the height direction as described above and, as shown in the upper view of FIG. 11, are arranged in a manner such that the inner surfaces bent and formed concave direct in the opposite directions to each other. Therefore, in a state that only the reflecting part 32A of the first float 30A floats up above the water surface of the salt water W as shown in the center view of FIG. 10, only a radio wave Ra from the right side of the view is reflected and only a reflected wave ra is generated, whereas a reflected wave of a radio wave Rb from the left side of the view is not generated. On the other hand, in a state that the reflecting part 32B of the second float 30B also floats up above the water surface of the salt water W as shown in the right view of FIG. 10, the reflected wave ra is generated from the first float 30A with respect to the radio wave Ra from the right side of the view, and a reflected wave rb is generated from the second float 30B with respect to the radio wave Rb from the left side of the view.

Then, with the configuration of the float described above, the receiving apparatus 20 and the measurement apparatus 10 can distinguish the receiving directions of the reflected waves r to measure the intensities thereof and thereby measure the abovementioned first salinity and second salinity in steps. For example, as shown in the center view of FIG. 10, when the reflected wave ra corresponding to the radio wave Ra from the right side of the view is received with an intensity equal to or more than a threshold value, the measurement apparatus 10 can measure that the first salinity is reached, and in addition, when the reflected wave rb corresponding to the radio wave Rb from the left side of the view is also received with an intensity equal to or more than the threshold value, the measurement apparatus 10 can measure that the second salinity is reached.

### Third Example Embodiment

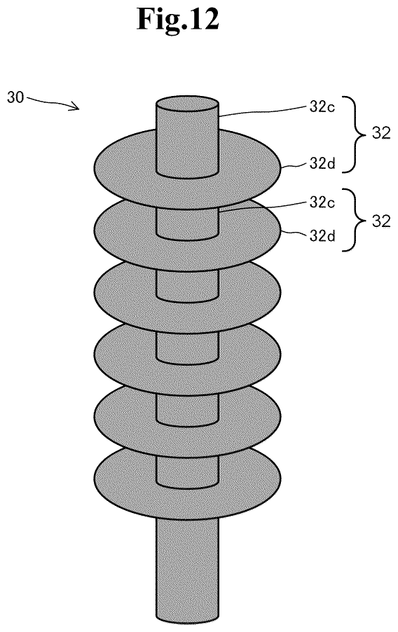

Next, a third example embodiment of the present disclosure will be described with reference to FIG. 12. FIG. 12 is a view for describing the configuration of a float in this example embodiment. In this example embodiment, a different configuration from the above example embodiments will be mainly described.

As shown in FIG. 12, the reflecting part 32 of the float 30 in this example embodiment is configured by a structure in which a cylindrical body 32c is placed at the center of a circular plate 32d and the surface thereof is made of a conductor surface as stated above. Then, the float 30 includes a plurality of reflecting parts 32 cyclically placed along the height direction of the float. That is to say, the float 30 in this example embodiment includes the reflecting parts 32 provided at a plurality of positions along the height direction. The float 30 is configured in a manner such that the floating height thereof increases as the salinity of the salt water W increases. Consequently, as the salinity of the salt water W increases, the number of the reflecting parts 32 floating up above the water surface increases.

When the intensity of the reflected wave r by the float 30 having the abovementioned configuration is measured, in accordance with the number of the reflecting parts 32 exposed on the water surface of the salt water W, a situation in which the reflected waves r by the respective reflecting parts 32 interfere with each other changes, and the intensity of the reflected wave r received and acquired by the receiving apparatus 20 changes so as to cyclically increase and decrease. For example, the intensity of the reflected wave r changes sinusoidally as the floating height increases. In this case, the measurement apparatus 10 can measure the salinity corresponding to the situation by measuring the cycle of strong and weak and the intensity of the acquired reflected wave r.

### Fourth Example Embodiment

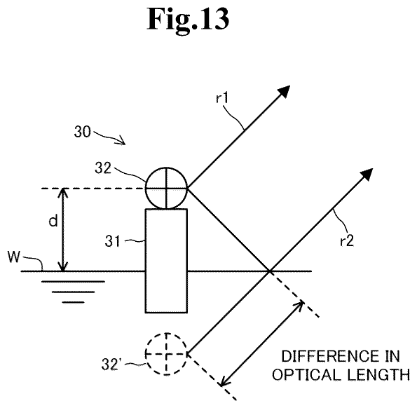

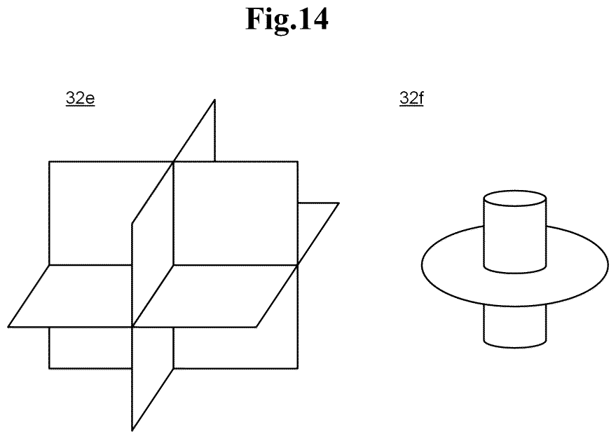

Next, a fourth example embodiment of the present disclosure will be described with reference to FIGS. 13 and 14. FIGS. 13 and 14 are views for describing the configuration of a float in this example embodiment. In this example embodiment, a different configuration from the above example embodiments will be mainly described.

As shown in FIG. 13, the float 30 in this example embodiment has the reflecting part 32 at the top end of the float main body 31. The reflecting part 32 in this example embodiment is configured to reflect the applied radio wave R to a direction in which the radio wave R enters, that is, upward, and to reflect the applied radio wave R toward the water surface of the salt water W. Specifically, the reflecting part 32 has, on the upper side, a first reflecting part that reflects the radio wave R upward and, on the lower side, a second reflecting part that reflects the radio wave R downward. For example, a reflecting part denoted by reference numeral 32e in FIG. 14 is configured by combining a corner reflector that opens upward and a corner reflector that opens downward. Moreover, for example, a reflecting part denoted by reference numeral 32f in FIG. 14 is configured by arranging a cylindrical body projecting upward and a cylindrical body projecting downward at the center of a circular plate.

With the reflecting part 32 of the float 30 having the configuration described above, as shown in FIG. 13, a first reflected wave r1 reflected by the first reflecting part placed on the upper side and a second reflected wave r2 reflected onto the water surface by the second reflecting part placed on the lower side and then reflected by the water surface as a mirror surface are generated. That is to say, the second reflected wave r2 by the mirror image of the reflecting part 32 denoted by reference numeral 32′ in FIG. 13 is also generated from the float 30. Consequently, as the height position of the float 30 changes, the state of interference between the first reflected wave r1 and the second reflected wave r2 changes, and the intensity of the reflected wave received and acquired by the receiving apparatus 20 changes so as to increase and decrease cyclically. The intensity of the reflected wave can be obtained by the following expressions, and changes sinusoidally as the floating height of the float 30 increases. In this case, the measurement apparatus 10 can measure the salinity corresponding to the situation by measuring the cycle of strong and weak and the intensity of the received and acquired reflected wave r.

Difference in optical path length=2d cos(incident angle)

Reflected wave=A₁ exp(2πjk(bias))+A₂ exp(2πjk(bias+difference in optical path length))

Square of reflected wave absolute value=const+A₁A₂ cos(2πjk optical path difference length)

A₁: amplitude of first reflected wave r1 reflected by first reflecting part

A₂: amplitude of second reflected wave r2 reflected by water surface as mirror surface

d: distance of reflecting part from water surface

bias: distance from satellite to reflecting part

j: imaginary unit

k: wave number of radar. However, since phase delay appears two times, once in a path from the transmitter to the reflecting part and once in a path from the reflecting part to the receiver, it is defined as 4π/wavelength of transmission signal of radar.

In the float 30 as described above, by covering a floating object such as a float main body located below the reflecting part 32 with a conductor, a reflected wave at the boundary between the reflecting part 32 and the floating object is also generated, and a change in the intensity of the reflected wave r can be increased. Moreover, by fixing a reflector on land or the like in addition to the float 30 as described above, the salinity can also be calculated based on comparison between the intensity of the reflected wave by the fixed reflector and the intensity of the reflected wave by the float 30. Furthermore, by arranging a plurality of floats 30 as described above in the salt water W and setting the shapes and densities of the respective floating objects so that the floating heights of the reflecting parts 32 of the respective floats 30 vary at the same salinity, the salinity can also be calculated based on comparison of the intensity changes of the reflected waves. In particular, by differentiating the vertical positions of the reflecting parts 32 in the respective floats 30, it is possible to configure a plurality of floats 30 that always produce a difference in “d: distance of reflecting part from water surface” described above. Since the square of reflected wave total value of the plurality of floats 30 cause phase-shifted sinusoidal changes, it is possible to more accurately measure whether d tends to increase or decrease by comparing them.

### Fifth Example Embodiment

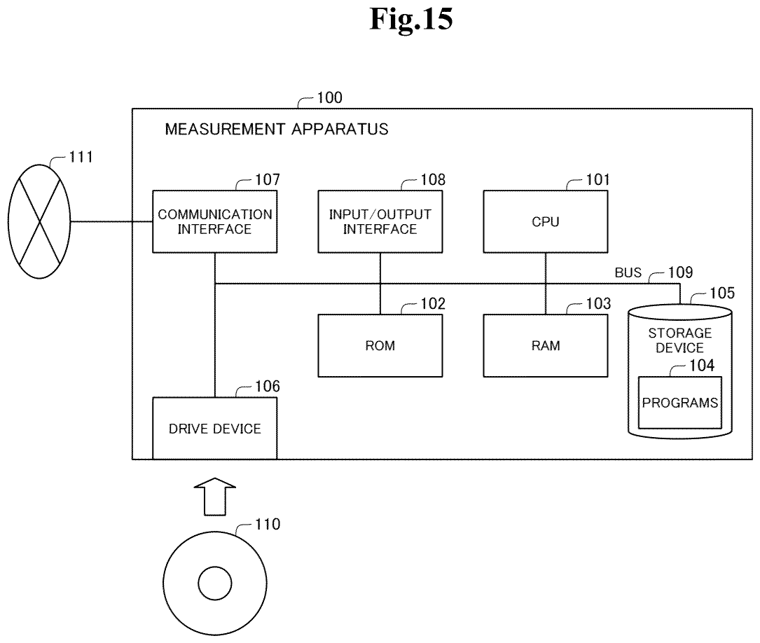

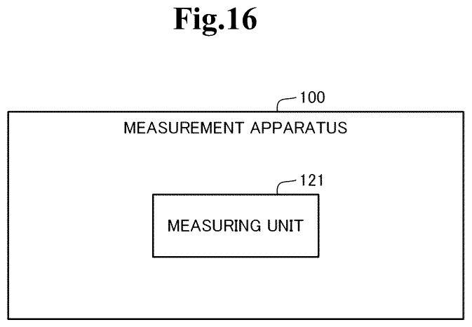

Next, a fifth example embodiment of the present disclosure will be described with reference to FIGS. 15 and 16. FIGS. 15 and 16 are block diagrams showing the configuration of a measurement apparatus in the fifth example embodiment. In this example embodiment, the overview of the configuration of the measurement apparatus described in the above example embodiments is shown.

First, with reference to FIG. 15, the hardware configuration of a measurement apparatus 100 in this example embodiment will be described. The measurement apparatus 100 is configured by a general information processing apparatus and, as an example, has a hardware configuration as shown below including
a CPU (Central Processing Unit) 101 (arithmetic logic unit),
a ROM (Read Only Memory) 102 (memory unit),
a RAM (Random Access Memory) 103 (memory unit),
programs 104 loaded to the RAM 103,
a storage device 105 storing the programs 104,
a drive device 106 reading from and writing into a storage medium 110 outside the information processing apparatus,
a communication interface 107 connected to a communication network 111 outside the information processing apparatus,
an input/output interface 108 performing input/output of data, and
a bus 109 connecting the components.

The measurement apparatus 100 can structure and include a measuring unit 121 shown in FIG. 16 by acquisition and execution of the programs 104 by the CPU 101. The programs 104 are, for example, stored in the storage device 105 or the ROM 102 in advance, and loaded to the RAM 103 and executed by the CPU 101 as necessary. The programs 104 may be delivered to the CPU 101 via the communication network 111, or may be stored in the storage medium 110 in advance and retrieved by the drive device 106 and delivered to the CPU 101. However, the abovementioned measuring unit 121 may be structured by a dedicated electronic circuit for realizing such means.

FIG. 15 shows an example of the hardware configuration of the information processing apparatus serving as the measurement apparatus 100, and the hardware configuration of the information processing apparatus is not limited to the abovementioned case. For example, the information processing apparatus may include some of the abovementioned components, such as not having the drive device 106. In addition, the information processing apparatus can use, instead of the CPU described above, a GPU (Graphic Processing Unit), a DSP (Digital Signal Processor), an MPU (Micro Processing Unit), an FPU (Floating point number Processing Unit), a PPU (Physics Processing Unit), a TPU (Tensor Processing Unit), a quantum processor, a microcontroller, or a combination thereof.

The measuring unit 121 measures the state of a liquid based on the intensity of a reflected wave of a radio wave applied to a float floating in the liquid. The float is configured in a manner such that a floating height thereof relative to the liquid changes in accordance with the state of the liquid, for example, the concentration, temperature, composition and so forth of the liquid and the intensity of the reflected wave changes in accordance with change in the floating height.

With the configuration as described above, the present disclosure can measure the state of a liquid such as concentration by measuring the intensity of a reflected wave of a radio wave applied to a float. Therefore, even when a liquid exists in a wide area, the state of the liquid can be measured with ease and with high accuracy.

The programs described above can be stored using various types of non-transitory computer-readable media and delivered to a computer. Non-transitory computer-readable media include various types of tangible storage media. Examples of non-transitory computer-readable media include a magnetic recording medium (for example, a flexible disc, a magnetic tape, a hard disk drive), a magneto-optical recording medium (for example, a magneto-optical disc), a CD-ROM (Read Only Memory), a CD-R, a CD-R/W, and a semiconductor memory (for example, a mask ROM, a PROM (Programmable ROM), an EPROM (Erasable PROM), a flash ROM, a RAM (Random Access Memory)). The program may also be delivered to a computer by various types of transitory computer readable media. Examples of transitory computer-readable media include an electrical signal, an optical signal, and an electromagnetic wave. Transitory computer-readable media can deliver the program to a computer via a wired channel such as a wire and an optical fiber, or a wireless channel.

Although the present disclosure has been described with reference to the above example embodiments and the like, the present disclosure is not limited to the above example embodiments. The configuration and details of the present disclosure can be changed in various manners that can be understood by a person skilled in the art within the scope of the present disclosure. At least one or more of the functions of the measuring unit 121 described above may be executed by an information processing apparatus installed in and connected to any location on the network, that is, may be executed by so-called cloud computing.

### Supplementary Notes

The whole or part of the above example embodiments can be described as the following supplementary notes. Below, the overview of configurations of a measurement system, a float, a measurement method, a measurement apparatus, and a program will be described. however, the present invention is not limited to the following configurations.

### Supplementary Note 1

A measurement system comprising:
a float configured to float in a liquid in a manner such that a floating height relative to the liquid changes in accordance with a state of the liquid; and
a measuring unit configured to measure the state of the liquid based on intensity of a reflected wave of a radio wave applied to the float,
wherein the float is configured in a manner such that the intensity of the reflected wave changes in accordance with a change in the floating height relative to the liquid.

### Supplementary Note 2

The measurement system according to Supplementary Note 1, wherein

the float includes a reflecting part installed in a predetermined position along a height direction and configured to reflect the radio wave, and a floating body configured to change a floating height position of the reflecting part relative to the liquid in accordance with a change in the state of the liquid.

### Supplementary Note 3

The measurement system according to Supplementary Note 2, wherein
the floating body of the float is configured to cause the reflecting part to float up above a liquid surface of the liquid when the liquid is in a specific state set in advance.

### Supplementary Note 4

The measurement system according to Supplementary Note 2, wherein

the reflecting part of the float is configured in a manner such that the intensity of the reflected wave is stronger as a position above a liquid surface of the liquid is higher.

### Supplementary Note 5

The measurement system according to Supplementary Note 2, wherein

the reflecting part of the float is configured in a manner such that the intensity of the reflected wave cyclically changes as a position above a liquid surface of the liquid is higher.

### Supplementary Note 6

The measurement system according to Supplementary Note 2, wherein

the float includes the reflecting part in each of a plurality of predetermined positions along the height direction.

### Supplementary Note 7

The measurement system according to Supplementary Note 2, wherein

the reflecting part of the float includes a first reflecting part configured to reflect the radio wave to a direction from which the radio wave has been applied, and a second reflecting part configured to reflect the radio wave toward a liquid surface of the liquid.

### Supplementary Note 8

The measurement system according to Supplementary Note 2, wherein:
the float includes at least two floats, a first float and a second float;
a first floating body included by the first float is configured to cause a first reflecting part included by the first float to float up above a liquid surface of the liquid when the liquid is in a first specific state set in advance;
a second floating body included by the second float is configured to cause a second reflecting part included by the second float to float up above the liquid surface of the liquid when the liquid is in a second specific state set in advance different from the first specific state; and
the first reflecting part and the second reflecting part are configured to reflect radio waves applied from mutually different directions.

### Supplementary Note 9

The measurement system according to Supplementary Note 1, wherein

the state of the liquid is concentration of the liquid.

### Supplementary Note 10

A float configured to float in a liquid in a manner such that a floating height relative to the liquid changes in accordance with a state of the liquid,

the float being further configured to reflect an applied radio wave in a manner such that intensity of a reflected wave changes in accordance with a change in the floating height relative to the liquid.

### Supplementary Note 11

The float according to Supplementary Note 10, comprising

a reflecting part installed in a predetermined position along a height direction and configured to reflect the radio wave, and a floating body configured to change a floating height position of the reflecting part relative to the liquid in accordance with a change in the state of the liquid.

### Supplementary Note 12

The float according to Supplementary Note 11, wherein

the floating body is configured to cause the reflecting part to float up above a liquid surface of the liquid when the liquid is in a specific state set in advance.

### Supplementary Note 13

The float according to Supplementary Note 11, wherein

the reflecting part is configured in a manner such that the intensity of the reflected wave is stronger as a position above a liquid surface of the liquid is higher.

### Supplementary Note 14

The float according to Supplementary Note 11, wherein

the reflecting part is configured in a manner such that the intensity of the reflected wave cyclically changes as a position above a liquid surface of the liquid is higher.

### Supplementary Note 15

The float according to Supplementary Note 11, comprising

the reflecting part in each of a plurality of predetermined positions along the height direction.

### Supplementary Note 16

The float according to Supplementary Note 11, wherein

the reflecting part includes a first reflecting part configured to reflect the radio wave to a direction from which the radio wave has been applied, and a second reflecting part configured to reflect the radio wave toward a liquid surface of the liquid.

### Supplementary Note 17

The float according to Supplementary Note 11, wherein:
the float includes at least two floats, a first float and a second float;
a first floating body included by the first float is configured to cause a first reflecting part included by the first float to float up above a liquid surface of the liquid when the liquid is in a first specific state set in advance;
a second floating body included by the second float is configured to cause a second reflecting part included by the second float to float up above the liquid surface of the liquid when the liquid is in a second specific state set in advance different from the first specific state; and
the first reflecting part and the second reflecting part are configured to reflect radio waves applied from mutually different directions.

### Supplementary Note 18

A measurement method comprising acquiring a reflected wave of a radio wave applied to a float and measuring a state of a liquid based on intensity of the acquired reflected wave, the float being configured to float in a liquid in a manner such that a floating height relative to the liquid changes in accordance with the state of the liquid and being further configured in a manner such that the intensity of the reflected wave of an applied radio wave changes in accordance with a change in the floating height relative to the liquid.

### Supplementary Note 19

A measurement apparatus comprising a measuring unit configured to acquire a reflected wave of a radio wave applied to a float and measure a state of a liquid based on intensity of the acquired reflected wave, the float being configured to float in the liquid in a manner such that a floating height relative to the liquid changes in accordance with the state of the liquid and being further configured in a manner such that the intensity of the reflected wave of the applied radio wave changes in accordance with a change in the floating height relative to the liquid.

### Supplementary Note 20

A non-transitory computer-readable medium storing a computer program, the computer program comprising instructions for causing a computer to execute processes to acquire a reflected wave of a radio wave applied to a float and measure a state of a liquid based on intensity of the acquired reflected wave, the float being configured to float in the liquid in a manner such that a floating height relative to the liquid changes in accordance with the state of the liquid and being further configured in a manner such that the intensity of the reflected wave of the applied radio wave changes in accordance with a change in the floating height relative to the liquid.

### DESCRIPTION OF NUMERALS

10 measurement apparatus
11 acquiring unit
12 measuring unit
16 criterion storing unit
20 receiving apparatus
30 float
31 float main body
32 reflecting part
A artificial satellite
W salt water
100 measurement apparatus
101 CPU
102 ROM
103 RAM
104 programs
105 storage device
106 drive device
107 communication interface
108 input/output interface
109 bus
110 storage medium
111 communication network
121 measuring unit

1. A measurement system comprising:
a float configured to float in a liquid in a manner such that a floating height relative to the liquid changes in accordance with a state of the liquid; and
a measuring unit configured to measure the state of the liquid based on intensity of a reflected wave of a radio wave applied to the float,
wherein the float is configured in a manner such that the intensity of the reflected wave changes in accordance with a change in the floating height relative to the liquid.

2. The measurement system according to claim 1, wherein
the float includes a reflecting part installed in a predetermined position along a height direction and configured to reflect the radio wave, and a floating body configured to change a floating height position of the reflecting part relative to the liquid in accordance with a change in the state of the liquid.

3. The measurement system according to claim 2, wherein
the floating body of the float is configured to cause the reflecting part to float up above a liquid surface of the liquid when the liquid is in a specific state set in advance.

4. The measurement system according to claim 2, wherein
the reflecting part of the float is configured in a manner such that the intensity of the reflected wave is stronger as a position above a liquid surface of the liquid is higher.

5. The measurement system according to claim 2, wherein
the reflecting part of the float is configured in a manner such that the intensity of the reflected wave cyclically changes as a position above a liquid surface of the liquid is higher.

6. The measurement system according to claim 2, wherein
the float includes the reflecting part in each of a plurality of predetermined positions along the height direction.

7. The measurement system according to claim 2, wherein
the reflecting part of the float includes a first reflecting part configured to reflect the radio wave to a direction from which the radio wave has been applied, and a second reflecting part configured to reflect the radio wave toward a liquid surface of the liquid.

8. The measurement system according to claim 2, wherein:
the float includes at least two floats, a first float and a second float;
a first floating body included by the first float is configured to cause a first reflecting part included by the first float to float up above a liquid surface of the liquid when the liquid is in a first specific state set in advance;
a second floating body included by the second float is configured to cause a second reflecting part included by the second float to float up above the liquid surface of the liquid when the liquid is in a second specific state set in advance different from the first specific state; and
the first reflecting part and the second reflecting part are configured to reflect radio waves applied from mutually different directions.

9. The measurement system according to claim 1, wherein
the state of the liquid is concentration of the liquid.

10. A float configured to float in a liquid in a manner such that a floating height relative to the liquid changes in accordance with a state of the liquid,
the float being further configured to reflect an applied radio wave in a manner such that intensity of a reflected wave changes in accordance with a change in the floating height relative to the liquid.

11. The float according to claim 10, comprising
a reflecting part installed in a predetermined position along a height direction and configured to reflect the radio wave, and a floating body configured to change a floating height position of the reflecting part relative to the liquid in accordance with a change in the state of the liquid.

12. The float according to claim 11, wherein
the floating body is configured to cause the reflecting part to float up above a liquid surface of the liquid when the liquid is in a specific state set in advance.

13. The float according to claim 11, wherein
the reflecting part is configured in a manner such that the intensity of the reflected wave is stronger as a position above a liquid surface of the liquid is higher.

14. The float according to claim 11, wherein
the reflecting part is configured in a manner such that the intensity of the reflected wave cyclically changes as a position above a liquid surface of the liquid is higher.

15. The float according to claim 11, comprising
the reflecting part in each of a plurality of predetermined positions along the height direction.

16. The float according to claim 11, wherein
the reflecting part includes a first reflecting part configured to reflect the radio wave to a direction from which the radio wave has been applied, and a second reflecting part configured to reflect the radio wave toward a liquid surface of the liquid.

17. The float according to claim 11, including at least two floats, a first float and a second float, wherein:
a first floating body included by the first float is configured to cause a first reflecting part included by the first float to float up above a liquid surface of the liquid when the liquid is in a first specific state set in advance;
a second floating body included by the second float is configured to cause a second reflecting part included by the second float to float up above the liquid surface of the liquid when the liquid is in a second specific state set in advance different from the first specific state; and
the first reflecting part and the second reflecting part are configured to reflect radio waves applied from mutually different directions.

18. A measurement method comprising acquiring a reflected wave of a radio wave applied to a float and measuring a state of a liquid based on intensity of the acquired reflected wave, the float being configured to float in a liquid in a manner such that a floating height relative to the liquid changes in accordance with the state of the liquid and being further configured in a manner such that the intensity of the reflected wave of an applied radio wave changes in accordance with a change in the floating height relative to the liquid.
